(19) United States
(12) Patent Application Publication
(10) Pub. No.: US 2021/0113456 A1
(43) Pub. Date: Apr. 22, 2021

(54) PHARMACEUTICAL COMPOSITIONS OF RAMELTEON AND METHODS OF USE THEREOF

(71) Applicant: MAXINASE LIFE SCIENCES LIMITED, Hong Kong (CN)

(72) Inventors: Yanfeng WANG, Hong Kong (CN); Liang XU, Hong Kong (CN); Juan CHEN, Hong Kong (CN); Benjamin Tak Kwong LEE, Hong Kong (CN)

(21) Appl. No.: 16/630,393

(22) PCT Filed: Jul. 12, 2018
(86) PCT No.: PCT/CN2018/095404

§ 371 Date: Jan. 10, 2020

Related U.S. Application Data
(60) Provisional application No. 62/532,266, filed on Jul. 13, 2017.

(51) Int. Cl. A61K 9/00; A61M 31/00; A61M 11/00; A61K 31/343; A61K 47/10; A61K 47/40; A61K 47/18; A61K 47/12; A61K 47/38; A61K 47/32

(52) U.S. Cl. CPC A61K 9/0043; A61M 2210/0618; A61M 11/007; A61K 9/006; A61K 31/343; A61K 47/10; A61K 47/40; A61K 47/183; A61K 47/186; A61K 47/12; A61K 47/38; A61K 47/32; A61K 9/0056; A61M 2210/0643; A61M 31/00

(57) ABSTRACT

The present invention relates to pharmaceutical compositions of ramelteon and methods of use thereof. More specifically, the present invention relates to pharmaceutical compositions of ramelteon for use in the treatment of insomnia and jet lag by transmucosal administration. The present invention achieved faster onset of pharmacological effects by delivery of low dosage of ramelteon via intranasal or sublingual routes, as compared to conventional oral tablet administration.

### FIELD OF THE INVENTION

The present invention relates to pharmaceutical compositions of ramelteon and methods of use thereof. More specifically, the present invention relates to pharmaceutical compositions of ramelteon for use in the treatment of insomnia and jet lag by transmucosal administration. The present invention achieved faster onset of pharmacological effects by delivery of low dosage of ramelteon via intranasal or sublingual routes, as compared to conventional oral tablet administration.

### BACKGROUND OF THE INVENTION

Approximately 30-40% of the population suffers from mild to severe insomnia. Insomnia may cause impairment of physical and metal health, such as fatigue and reduced alertness, irritability, and impaired concentration, with increasing risk for accidents and reduced work productivity. All these symptoms have a negative impact on the quality of life. Appropriate treatments for insomnia include lifestyle modification, sleep hygiene, and pharmacologic interventions by sedative-hypnotics.

Unlike traditional hypnotics acting at the GABA receptor complex, ramelteon has a novel mechanism of action and is the only melatonin agonist for the treatment of insomnia. Similar to melatonin, an endogenous hormone synthesized in the pineal gland, ramelteon can effectively induce and prompt natural sleep by shorting the latency to sleep onset and by increasing the total sleep time. Ramelteon acts on the MT1 and MT2 receptors only and demonstrates no affinity for any other CNS receptors associated with sedation (e.g., GABA, dopamine, opiate, serotonin). Therefore, ramelteon has low potential for abuse and is not classified as a scheduled drug by US Drug Enforcement Agency (DEA). Clinical trials show the treatment with ramelteon is well tolerated with few adverse effects and is suitable for long-term use up to 6 months. Moreover, age and gender do not impact ramelteon's sedative properties.

Despite the aforementioned advantages, oral ramelteon shows very low bioavailability (1.8%) due to the extensive first-pass metabolism in the liver, and ramelteon is metabolized by multiple cytochrome P-450 (CYP450) isoenzymes, including CYP1A2, CYP2C subfamily, and CYP3A4. Therefore, high inter-subject variability (up to 100%) and significant drug-drug interactions can be expected. For example, after co-administration fluvoxamine, a strong CYP1A2 inhibitor, the systemic exposure (AUC) for ramelteon increased approximately 190-fold. Other CYP450 inhibitors or inducers, like rifampin, ketoconazole, fluconazole, donepezil and doxepin, can also significantly alter ramelteon AUC and peak plasma concentration (Cₘₐₓ) in human trials. Besides, the systemic exposure of ramelteon increased in the hepatic impairment patients (8- to 10.7-fold higher AUCs) relative to healthy volunteers. Food can also result in higher AUC but delayed time to Cₘₐₓ (Tₘₐₓ).

Another safety concern on oral ramelteon is the active M-II metabolite after hydroxylation. M-II has about one-tenth and one-fifth binding affinity for the MT1 and MT2 receptors, respectively. However, the systemic exposure to M-II is 20- to 100-fold greater than the parent drug itself. Therefore, M-II should substantially contribute to the total pharmacological effects, and potentially results in next-morning residual effects (i.e., drowsiness, memory impairment) with respect to its long elimination half-life and extend persistence in the circulation. Moreover, the high concentration M-II was found to increase the incidence of hepatic tumors in animal chronic toxicology study.

Therefore, even though ramelteon is well known in the art, several disadvantages nevertheless remain. Consequently, there is still a need for improved compositions and methods for ramelteon, particularly as they relate to compositions and methods that avoid rapid metabolic degradation.

### SUMMARY OF THE INVENTION

One aspect of the inventive subject matter relates to pharmaceutical compositions comprising ramelteon in a suitable formulation at a dose of 0.1 to 50 mg. The composition is suitable for transmucosal administration, typically to administer through intranasal or sublingual mucosa.

The use of ramelteon according to the invention includes formulations wherein the treatment dosage of ramelteon is delivered to the mucosal membrane. The preferred formulations are in liquid dosage forms, including a solution, suspension, emulsion, bioadhesive or in situ gels, microsphere, nanoparticle, self-emulsifying drug delivery system, soft gel capsule; or in solid dosage forms, including powders, granules, capsules, pills, tablets, lozenges and lollipop; or in semi-solid dosage forms, including ointments, creams, hydrogel, films and patches; or other forms suitable for transmucosal delivery known in the art.

A further aspect of the invention relates to use of a treatment dosage of ramelteon comprising 0.1 to 50 mg ramelteon in a suitable pharmaceutical vehicle for transmucosal delivery, for preparation of a medicament for treatment of insomnia, or for relief of jet lag and related symptoms.

One aspect of the inventive subject matter relates to a pharmaceutical composition for treating insomnia or jet lag, by transmucosal administration, comprising 0.01% to 10% by weight of ramelteon. According to one aspect of the present invention, said the pharmaceutical composition is a liquid solution comprising: 0.01% to 2% (w/v) ramelteon; 5% to 30% (w/v) propylene glycol; 5% to 60% (w/v) sulfobutyl ether-β-cyclodextrin; 0.01% to 1% (w/v) EDTA.2Na; and 0.01% to 0.1% (w/v) benzalkonium chloride.

According to one aspect of the present invention, said pharmaceutical composition is formulated into nasal spray or nasal drop and for intranasal administration in mammals. According to one aspect of the present invention, said intranasal administration is completed by an intranasal delivery system, the intranasal delivery system comprises a bottle and metered multi-dose pump. According to one aspect of the present invention, said pharmaceutical composition is formulated to deliver a volume of said composition of 0.05 ml to 0.15 ml per intranasally delivery. According to one aspect of the present invention, said pharmaceutical composition is formulated to deliver a dose of 0.05 to 25 mg ramelteon per intranasally delivery.

According to one aspect of the present invention, said pharmaceutical composition is formulated into sublingual spray for sublingual administration in mammals. According to one aspect of the present invention, said pharmaceutical composition is administrated by a sublingual delivery system, said sublingual delivery system comprises a bottle and metered multi-dose pump. According to one aspect of the present invention, said pharmaceutical composition is formulated to sublingually deliver a volume of said composition of 0.07 ml to 0.25 ml per delivery. According to one aspect of the present invention, said pharmaceutical composition is formulated to sublingually deliver a dose of 0.1 to 50 mg ramelteon per delivery. According to one aspect of the present invention, said pharmaceutical composition formulated into sublingual spray is a liquid solution comprising: 0.01% to 2% (w/v) ramelteon; 5% to 80% (w/v) glycerol monooleate; 2% to 50% (w/v) ethanol.

According to one aspect of the present invention, said pharmaceutical compositions can be further formulated into suspension, emulsion, bioadhesive or in-situ gel, microsphere, nanoparticle, self-emulsifying drug delivery system and soft gel capsule. According to one aspect of the present invention, said the pharmaceutical composition is a solid dosage form comprising 0.1%-10% by weight of ramelteon and at least 90% of excipients, wherein said excipients selected from HP-beta-cyclodextrin, crospovidone, croscarmellose sodium, microcrystalline cellulose, colloidal silicon dioxide, sodium starch glycolate, sodium stearyl fumarate, lacotose, corn starch and/or magnesium stearate. According to one aspect of the present invention, the pharmaceutical composition is formulated into a solid tablet suitable for sublingual administration. According to one aspect of the present invention, the tablet has a disintegration time of less than 15 min. According to one aspect of the present invention, said solid dosage form is a film or a strip and wherein said pharmaceutical composition comprises a mucoadhesive polymer.

### BRIEF DESCRIPTION OF THE DRAWING

FIG. 1 is a graph showing the dissolution rate of tablets prepared by wet granulation or direct compression.

FIG. 2 is a graph illustrating mean ramelteon plasma concentration versus time profiles after intranasal administration of RNS-01 and oral administration of ROS-01 at a single dose of 0.1 mg/rat.

FIG. 3 is a graph illustrating mean ramelteon plasma concentration vs. time profiles in beagle dogs receiving a single sublingual dose of 2 mg sublingual liquid spray or a single oral dose of 8 mg Rozerem®.

FIG. 4 is a graph showing ramelteon mean plasma concentration vs. time profiles in male beagle dogs receiving a single sublingual dose of 1 mg tablet, or a single sublingual dose of 1 mg film, or a single sublingual dose of 1 mg LC spray or a single oral dose of 8 mg Rozerem®.

### DETAILED DESCRIPTION OF THE INVENTION

The invention relates to the pharmaceutical compositions capable of delivering sufficient dose of ramelteon through transmucosal routes, including but not limited to intranasal or sublingual routes. The conventional oral administration of ramelteon results in various undesirable effects, such as high first-pass effect with very low bioavailability, high inter-subject variation, drug-drug interactions and food effect, delayed sleep-inducting effect, as well as potential adverse effects caused by major active metabolites. The present transmucosal compositions enable ramelteon to be quickly absorbed through the nasal mucosa or sublingual mucosa, thereby advantageously bypassing the metabolism in the GI tract and liver. Consequently, most if not all disadvantages of oral administration can be well addressed with improved absorption, rapid onset, and/or minimal side effects.

Viewed from a different perspective, drug delivery via transmucosal delivery routes, including intranasal and sublingual, may be alternative and promising choices since ramelteon can directly enter into the blood circulation from the absorption sites. Thus, high first-pass metabolism in the liver can be completely bypassed. Ramelteon is small organic molecule with high lipid solubility, and is an ideal drug candidate for transmucosal delivery. In vitro results showed the high permeability through Caco-2 monolayer, implying the fast and complete absorption from nasal and sublingual areas.

The advantage of transmucosal compositions of ramelteon include rapid sleep onset with prolonged sleep maintenance; bypass first-pass metabolism and high bioavailability; dose reduction with less next-morning residual effects; minimal metabolite concentration in circulation and related adverse effects; minimal drug-drug interaction and food effect, minimal inter-subject variation; no dose adjustment required in special populations, i.e. hepatic impairment patients; excellent safety profiles; and ease of use.

Surprisingly, the inventors found that the bioavailability of ramelteon can be improved over 20-fold in rats after intranasal administration of the pharmaceutical compositions described in the present invention, as compared to the oral administration of the drug solution with same dose. According to the inventors' knowledge, there is no patent or publication showing the delivery of ramelteon via transmucosal routes, i.e. intranasal and sublingual, with the unexpected rapid and complete adsorption over the conventional oral tablet. Based on the animal pharmacokinetic results, the intranasal or sublingual dose can therefore be reduced, but systemic absorption will be comparable to the oral tablet of the same dose, while maintaining or even improving onset of sleep, minimal toxic metabolite concentrations, food effects, and/or drug-drug interactions.

### EMBODIMENTS

### Example 1

### Ramelteon Nasal Spray

In one exemplary composition of the invention, a nasal composition was prepared using the solubilisers like Propylene Glycol and Sulfobutyl ether-β-Cyclodextrin (SBE-CD). Firstly, 5 g Ramelteon was completely dissolved in 150 mL propylene glycol, the solution was then gradually added into 5 mL SBE solution (52%, w/v) containing EDTA.2Na and Benzalkonium Chloride, finally q.s purified water was added to 1 L.

| Ingredients | Amount | Unit |
|---|---|---|
| Ramelteon | 5 | g |
| Propylene Glycol | 0.15 | L |
| Sulfobutyl ether-β-Cyclodextrin | 260 | g |
| EDTA•2Na | 0.2 | g |
| Benzalkonium Chloride | 0.125 | g |

| | |
|---|---|
| Purified Water | Q.S. to 1 L |

Following preparation, the drug solution was filtered through 0.22 μm filter membrane and was then filled into a glass bottle fitted with a metered-dose spray pump for intranasal application in a volume of 0.10 ml/spay. 0.5 mg ramelteon will be delivered intranasally per spray.

### Example 2

Ramelteon Sublingual Tablet

Ramelteon sublingual tablets were prepared by two different methods: direct compression and wet granulation. For direct compression process, ramelteon was mixed with excipients. The physical mixture was compressed directly with a single punch tableting machine (Shanghai Tianfan pharmaceutical machine factory, type 6A). The wet granulation processes are listed below:
1. Weighing the ingredients according to the formula.
2. Dissolve ramelteon, HP-β-cyclodextrin into the ethanol under stirring.
3. Mix ramelteon-cyclodextrin solution with Kollidon® CL-SF to granulate. The wet material is passed through a 40 mesh sieve.
4. Drying: drying the wet granules at 60° C. for 120 min in oven.
5. Dry sieving: the dry granules were milled and sieved through a 60 mesh sieve.
6. Final blending: extra-granular ingredients (PROSOLV® EASYtab SP and magnesium stearate) are added into the dry granules. The final granules are blended by hand.
7. Tablet compressing: strength: 2 mg/tab. Hardness: 4˜8 kgf. Tablet shape: round biconvex tablet (Diameter: 6 mm).

| Ingredients | mg/tab | g/1000 tab |
|---|---|---|
| Ramelteon | 2 | 2 |
| HP-β-CD | 50 | 50 |
| Kollidon ® CL-SF | 40 | 40 |
| PROSOLV ® EASYtab SP | 26.9 | 26.9 |
| Magnesium stearate | 1.1 | 1.1 |
| Tablet weight | 120 | 120 |

### Example 3

### Dissolution Test on Ramelteon Sublingual Tablet

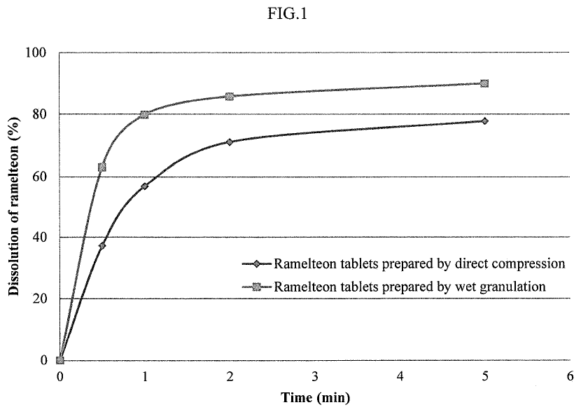

Dissolution test was performed using a dissolution tester (TIANDA TIANFA-pharmaceutical Testing Instrument Manufacturer, ZRS-8L) in accordance with USP 36/NF 31 dissolution test apparatus II (Paddle). 100 mL of purified water was taken as the dissolution medium at 37° C. The paddle speed is kept at 50 rpm. 2 mL of aliquots were withdrawn at fix time intervals, and the sample volume was replaced by an equal volume of purified water. The dissolution samples were analyzed with HPLC. The dissolution profiles of ramelteon tablets prepared by direct compression and wet granulation are shown in FIG. 1, the dissolution rate of wet granulation tablets is faster than tablets prepared with direct compression.

### Example 4

### Ramelteon Mucoadhesive Sublingual Spray

Ramelteon mucoadhesive formulation was prepared by dissolving ramelteon in mucoadhesive vehicles consisting of glyceryl monooleate (GMO), organic solvents, surfactants and preservatives. The preparation method is summarized below:
1. Dissolve GMO in Cremophor EL at 40° C.˜50° C.
2. Add ethanol into the mixture in step 1.
3. Dissolve ramelteon in the mixture solution in step 2.
4. Add benzalconium chloride into the ramelteon solution in step 3 and mixing.

| Ingredients | Proportion (%) | mg/unit |
|---|---|---|
| Ramelteon | 0.99 | 2 |
| Cremophor ® EL | 60.08 | 121.7 |
| Glyceryl monooleate | 25.75 | 52.2 |
| Ethanol | 12.87 | 26.1 |
| Benzalkonium chloride | 0.31 | 0.626 |
| Total | 100 | 202.6 |

The formulation is filled into a metered dose sublingual spray kit with a total volume of 5 mL, each actuation (0.1 mL) can provide a dose of 2 mg ramelteon sublingually.

### Example 5

### Ramelteon Fast-Dissolving Sublingual Film

Sublingual films were prepared by solvent casting method. HPMC E5 and E15 were selected as film-forming polymers and PEG 400 was used as a plasticizer. Aqueous solution 1 was prepared by dissolving the polymers in boiled and distilled water and stirred at room temperature for 1 hour until the polymers swell. Solution 2 was prepared by dissolving ramelteon and HP-beta-Cyclodextrin at pre-determined ratio in ethanol (absolute). Solution 1 and solution 2 were then mixed up and then PEG 400 was added into the solution. After sonicating for 20 min to remove the air bubble, the mixture solution was casted onto a plastic petri dish and was dried in the oven at 60° C. for 5 h. The film was carefully removed from the petri dish, checked for any imperfections, and cut into square size (2.0 cm length, 2.0 cm width). The samples were stored in a glass desiccator for further analysis.

Different formulations were prepared by HPMC E5, HPMC E15, or a combination of HPMC E5 and HPMC E15, the film forming capacity and their effect on the physicochemical properties were studied. Different ratios of polymer to plasticizer and ramelteon to HP-beta-Cyclodextrin were also evaluated. Disintegrant was used in order to decrease the disintegration time.

| | | | | | HPMC E15 | | |
|---|---|---|---|---|---|---|---|
| Formulation | | HP-beta- | HPMC | HPMC | & E5 | PEG | Starch |
| Code | Ram | Cyclodextrin | E15 | E5 | (1:1) | 400* | 1500 |
| 170809-2 | 1 | 25 | 24 | — | — | 1.5 | — |
| 170817-1 | 1 | 25 | — | 24 | — | 1.5 | — |
| 170814-1 | 1 | 25 | 15 | — | — | 1.5 | — |
| 170821-1 | 1 | 25 | — | — | 16 | 1.5 | — |
| 170822-1 | 1 | 25 | — | — | 16 | 1.5 | 3.5 |
| 170831-1 | 1 | 25 | 8 | — | — | 1.5 | — |
| 170831-2 | 1 | 35 | 8 | — | — | 1.5 | — |
| 170901-1 | 1 | 15 | 8 | — | — | 2.0 | — |
| 170901-2 | 1 | 15 | 8 | — | — | 2.5 | — |
| 170904-1 | 1 | 15 | 8 | — | — | 1.8 | — |

*Polymer: PEG 400 = 1:1.5/2.0/2.5/1.8;

### Example 6

### In-Vitro Drug Release Test on Ramelteon Sublingual Film

Since the film is dissolve sublingually in presence of limited volume of saliva, 2 mL of medium was used to evaluate its dissolution. A strip was placed in a centrifuge tube containing 2 mL of purified water and then vortexed for 5 min until homogeneous solution was obtained, the solution was then filtered and the filtrate was analysed after appropriate dilution. The dissolution of film was tested in triplicate.

As shown in table below, the film with 2 mg ramelteon can be dissolved in 2 mL of purified water due to the solubilization effect by HP-beta-Cyclodextrin.

| | Weight | | Released Amount | Dissolution |
|---|---|---|---|---|
| Sample | (mg) | Area | (mg) | (%) |
| F1 | 82.4 | 1322492 | 1.866 | 96.64 |
| F2 | 83.8 | 1332084 | 1.880 | 95.71 |
| F3 | 84.3 | 1362067 | 1.922 | 97.29 |

### Example 7

### Pharmacokinetics of Ramelteon after Nasal and Oral Administration

The objective was to study the pharmacokinetics of Ramelteon by determining the plasma concentrations after oral and intranasal administration. SD rats (n=3-4 for each dose) received an intranasal dose (0.1 mg/animal) of nasal solution composition (RNS-01, 2.5 mg/mL) which was prepared according to the EXAMPLE 1 of this invention, and an oral dose (0.1 mg/animal) of Granisetron oral solution (ROS-01) which was prepared by directly dissolving ramelteon into saline solution to the final concentration of 0.1 mg/mL. Multiple blood samples were collected from tail vein until 6 hrs. Ramelteon concentration in rat plasma was determined using a validated LC/MS/MS method. The standard non-compartmental method was used to generate the pharmacokinetic parameters.

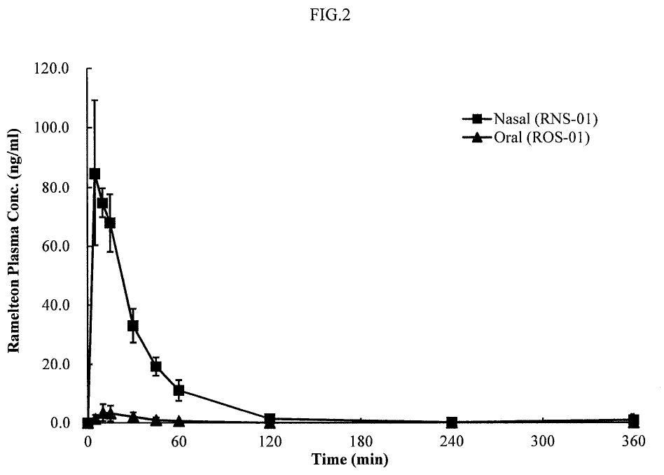

FIG. 2 shows the mean ramelteon plasma concentration versus time profiles after intranasal administration of RNS-01 and oral administration of ROS-01 at a single dose of 0.1 mg/rat. Results indicate the intranasal dose can achieve significantly higher and prolonged ramelteon plasma concentration than the same oral dose of solution composition.

### Example 8

### Pharmacokinetics Study of Ramelteon Oral Tablet and Sublingual Spray in Beagle Dogs

The objective of this study was to assess the pharmacokinetics of ramelteon after oral administration of Rozerem® tablet and sublingual administration of liquid spray in beagle dogs. A total of 6 beagle dogs (3 male/3 female) was used in this pharmacokinetic study. In period I, a single oral dose of 8 mg ramelteon tablet (Rozerem®) was administered to male dogs under fasted condition, and a single sublingual dose of 2 mg ramelteon liquid spray (1 mg/spray, total 2 sprays) was given to female dogs. In period II, a single oral dose of 8 mg ramelteon tablet (Rozerem®) was administered to female dogs under fasted condition, and a single sublingual dose of 2 mg ramelteon liquid spray (1 mg/spray, total 2 sprays) was given to male dogs. The concentration of ramelteon in plasma samples were quantified using a validated LC-MS/MS method. Pharmacokinetic parameters were calculated by non-compartmental model.

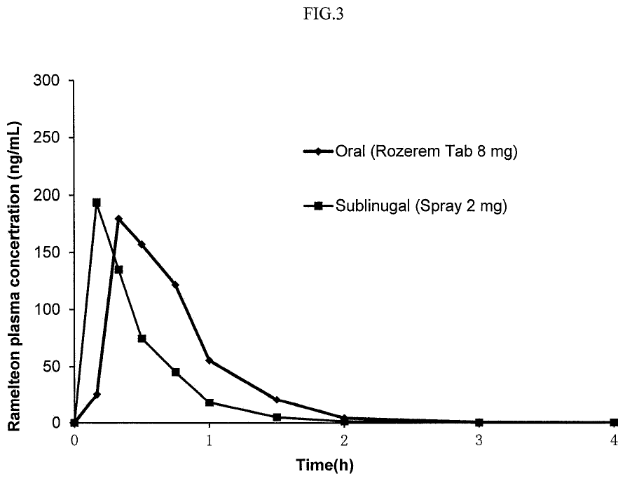

FIG. 3 shows Ramelteon mean plasma concentration vs. time profiles in beagle dogs receiving a single sublingual dose of 2 mg (sublingual spray), or a single oral dose of 8 mg (Rozerem®) (n=6). The mean Tₘₐₓ is 0.194 h after sublingual dose of 2 mg, as compared to 0.568 h after oral dose of 8 mg, indicating the faster drug absorption after sublingual administration in dogs. The relative bioavailability of ramelteon sublingual spray to oral tablet is 279%, suggesting the improved absorption from sublingual route as compared to oral route.

### Example 9

### Pharmacokinetics Study of Ramelteon Sublingual Formulations in Male Beagle Dogs

A total of 12 beagle dogs were randomly divided into 3 groups. Group 1 (n=4) was given a single sublingual dose of 1 mg ramelteon sublingual tablet. Group 2 (n=4) was sublingually dosed with 1 mg ramelteon oral film. Group 3 (n=4) was sublingually administrated with 1 mg ramelteon mucoadhesive (LC) spray. Blood samples (about 2 mL) were collected at pre-dose, and 0.167, 0.33, 0.5, 0.75, 1, 1.5, 2, 4, 6, 8, 12, and 24 h post-dose using EDTA-K₂ as anticoagulant. The blood samples were centrifuged at 4,000 rpm for 10 min to obtain plasma samples. The plasma samples were stored at −80° C. for bioanalysis. The concentration of ramelteon in plasma samples were quantified using a validated LC-MS/MS method. Pharmacokinetic parameters were calculated by non-compartmental model.

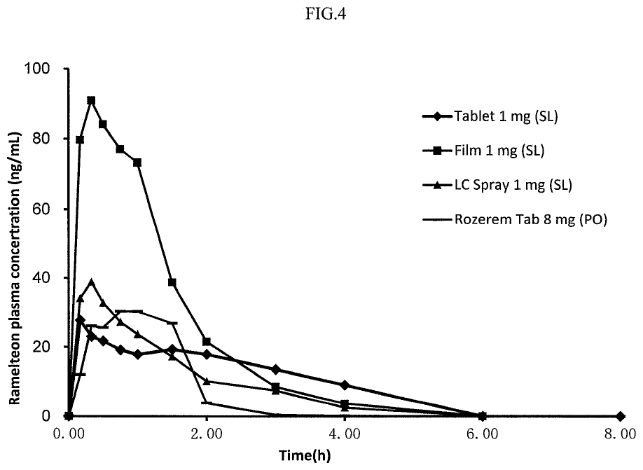

FIG. 4 shows Ramelteon mean plasma concentration vs. time profiles in male beagle dogs receiving a single sublingual dose of 1 mg tablet, or a single sublingual dose of 1 mg film, or a single sublingual dose of 1 mg LC spray (n=4), as compare to that after a single oral dose of 8 mg (Rozerem®) (n=3). Results indicate the faster drug absorption after sublingual administration of all 3 formulations as compared to oral administration of marketed product (Rozerem®) in dogs. PK parameters in the table below suggest that 1 mg sublingual dose can achieve comparable plasma concentration to 8 mg Rozerem® oral tablet.

| PK | | Formulation (Route) | | | |
|---|---|---|---|---|---|
| Parameters | Unit | Tablet (SL) | Film (SL) | Spray (SL) | Rozerm ® (PO) |
| Tₘₐₓ | h | 2.29 ± 1.64 | 0.33 ± 0.00 | 0.39 ± 0.10 | 1.08 ± 0.38 |
| Cₘₐₓ | μg/L | 36.98 ± 15.13 | 90.97 ± 16.80 | 38.83 ± 1.19 | 42.10 ± 31.69 |
| AUC₀₋ₜ | μg/L*h | 76.0 ± 8.63 | 141.34 ± 21.18 | 62.05 ± 11.42 | 145.38 ± 113.59 |
| AUC_{0-inf} | μg/L*h | 76.0 ± 8.63 | 142.32 ± 20.06 | 62.50 ± 10.84 | 145.59 ± 113.75 |
| T_{1/2} | h | 0.45 ± 0.12 | 0.51 ± 0.13 | 0.52 ± 0.16 | 0.94 ± 0.16 |
| MRT | h | 2.08 ± 0.69 | 1.17 ± 0.17 | 1.36 ± 0.32 | 1.19 ± 0.45 |

1. A pharmaceutical composition for treating insomnia or jet lag, by transmucosal administration, comprising 0.01% to 50% by weight of ramelteon.

2. The pharmaceutical composition of claim 1, comprising 0.01% to 10% by weight of ramelteon.

3. The pharmaceutical composition of claim 1, wherein said the pharmaceutical composition is a liquid solution comprising:
about 0.01% to about 2% (w/v) ramelteon;
about 5% to 30% (w/v) propylene glycol;
about 5% to 60% (w/v) sulfobutyl ether-β-cyclodextrin;
about 0.01% to 1% (w/v) EDTA.2Na;
about 0.01% to 0.1% (w/v) benzalkonium chloride.

4. The pharmaceutical composition of claim 1, wherein said pharmaceutical composition is formulated into nasal spray or nasal drop and for intranasal administration in mammals.

5. The pharmaceutical composition of claim 4, wherein said intranasal administration is completed by an intranasal delivery system, the intranasal delivery system comprises a bottle and metered multi-dose pump.

6. The pharmaceutical composition of claim 4, wherein the said pharmaceutical composition is formulated to deliver a volume of said composition of about 0.05 ml to 0.15 ml per intranasally delivery.

7. The pharmaceutical composition of claim 4, wherein the said pharmaceutical composition is formulated to deliver a dose of 0.05 to 25 mg ramelteon per intranasally delivery.

8. The pharmaceutical composition of claim 1, wherein said pharmaceutical composition is formulated into sublingual spray for sublingual administration in mammals.

9. The pharmaceutical composition of claim 8, wherein said pharmaceutical composition is administrated by a sublingual delivery system, said sublingual delivery system comprises a bottle and metered multi-dose pump.

10. The pharmaceutical composition of claim 8, wherein the said pharmaceutical composition is formulated to sublingually deliver a volume of said composition of about 0.07 ml to 0.25 ml per delivery.

11. The pharmaceutical composition of claim 8, wherein the said pharmaceutical composition is formulated to sublingually deliver a dose of 0.1 to 50 mg ramelteon per delivery.

12. The pharmaceutical composition of claim 8, wherein said the pharmaceutical composition is a liquid solution comprising:
about 0.01% to about 2% (w/v) ramelteon;
about 5% to 80% (w/v) glycerol monooleate;
about 2% to 50% (w/v) ethanol.

13. The pharmaceutical composition of claim 1, wherein said pharmaceutical compositions can be further formulated into suspension, emulsion, bioadhesive or in-situ gel, microsphere, nanoparticle, self-emulsifying drug delivery system and soft gel capsule.

14. The pharmaceutical composition of claim 1, wherein said the pharmaceutical composition is a solid dosage form comprising 0.1%-10% by weight of ramelteon and at least 90% of excipients, wherein said excipients selected from HP-beta-cyclodextrin, crospovidone, croscarmellose sodium, microcrystalline cellulose, colloidal silicon dioxide, sodium starch glycolate, sodium stearyl fumarate, lacotose, corn starch and/or magnesium stearate.

15. The pharmaceutical composition of claim 14, which is formulated into a solid tablet suitable for sublingual administration.

16. The pharmaceutical composition of claim 15, wherein the tablet has a disintegration time of less than 15 min.

17. The pharmaceutical composition of claim 14, wherein said solid dosage form is a film or a strip and wherein said pharmaceutical composition comprises a mucoadhesive polymer.
